(19) United States
(12) Patent Application Publication
(10) Pub. No.: US 2018/0153509 A1
(43) Pub. Date: Jun. 7, 2018

(54) ULTRASOUND IMAGING WITH SMALL-ANGLE ADJUSTMENT

(71) Applicant: General Electric Company, Schenectady, NY (US)

(72) Inventors: Andreas Kremsl, Wolfgang im Salzkammergut (AT); Manuel Schoenauer, Thalgau (AT); Reinhold Bruestle, Frankenburg am Hausruck (AT)

(21) Appl. No.: 15/370,961

(22) Filed: Dec. 6, 2016

(51) Int. Cl. A61B 8/00; G01S 7/52; G01S 15/89; A61B 8/14

(52) U.S. Cl. CPC A61B 8/4466; G01S 7/52079; G01S 15/8915; A61B 8/469; A61B 8/145; A61B 8/54; A61B 8/4494

(57) ABSTRACT

Methods and systems are provided for an ultrasound probe including a small-angle adjustment element. In one embodiment, the small-angle adjustment element includes a piezoelectric actuator configured to rotate a transducer array of the ultrasound probe in response to electrical signals transmitted to the piezoelectric actuator. In this way, an amount of angle spanned by the transducer array during a scan of a region of interest may be reduced, thereby increasing a precision of the ultrasound probe.

### FIELD

Embodiments of the subject matter disclosed herein relate to ultrasound imaging, and more particularly, to ultrasound probes.

### BACKGROUND

An ultrasound imaging system may include an ultrasound probe configured to generate sound wave pulses and detect returning echoes within an object via an array of transducers. Such transducers typically include electromechanical elements capable of converting electrical energy into mechanical energy for transmission of ultrasonic wave pulses into patient tissue. Mechanical energy of reflected ultrasonic waves is then converted back into electrical energy when the reflected ultrasonic waves reach the transducers. The probe may be positioned by an operator of the system to scan a desired region of interest (e.g., a desired tissue or body region to be imaged), and echoes returning to the probe during the scan are processed into an image by a workstation or computational device of the system which may then display the image via a display device.

In some examples, the transducer array may be rotated by an actuator such as a stepper motor in order to acquire a sequence of images across a volume. The workstation or computational device may then compare images within the sequence to each other to produce a compound image with reduced acoustical artifacts in a process referred to as volume compound imaging (VCI).

However, an image sequence acquired via rotation of the transducer array by the stepper motor may span a large angle. As a result, a given image plane may span a relatively large amount of distance from adjacent image planes, thereby reducing an amount of acoustical artifact reduction provided by VCI and decreasing a scanning precision of the ultrasound probe. Therefore, a reduction in the distance between each image plane may increase a quality of images produced by the ultrasound system.

### BRIEF DESCRIPTION

In one embodiment, an ultrasound probe comprises: a sensor housing; a piezoelectric actuator disposed within the sensor housing; and a transducer array coupled with the sensor housing, the transducer array rotatable relative to the sensor housing via energization of the piezoelectric actuator. In this way, the piezoelectric actuator may be energized to rotate the transducer array by a small angle, thereby increasing a scanning precision of the ultrasound probe.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a partial cross-sectional view of an ultrasound probe including a large-angle adjustment element and a small-angle adjustment element.

FIG. 2 shows the ultrasound probe in various partial cross-sectional views, with a transducer array of the probe rotated by the large-angle adjustment element.

FIG. 3 shows a first embodiment and a second embodiment of the small-angle adjustment element in cross-sectional views.

FIG. 4 shows various signal waveforms that may actuate the small-angle adjustment element.

FIG. 5 shows the ultrasound probe in various partial cross-sectional views, with a transducer array of the probe rotated by the small-angle adjustment element.

FIG. 6 shows an example method of acquiring images across a small angle by actuating the small-angle adjustment element of the ultrasound probe.

FIG. 7 shows an example method of acquiring images across a large angle by actuating the large-angle adjustment element of the ultrasound probe, and enhancing image acquisition by actuating the small-angle adjustment element.

### DETAILED DESCRIPTION

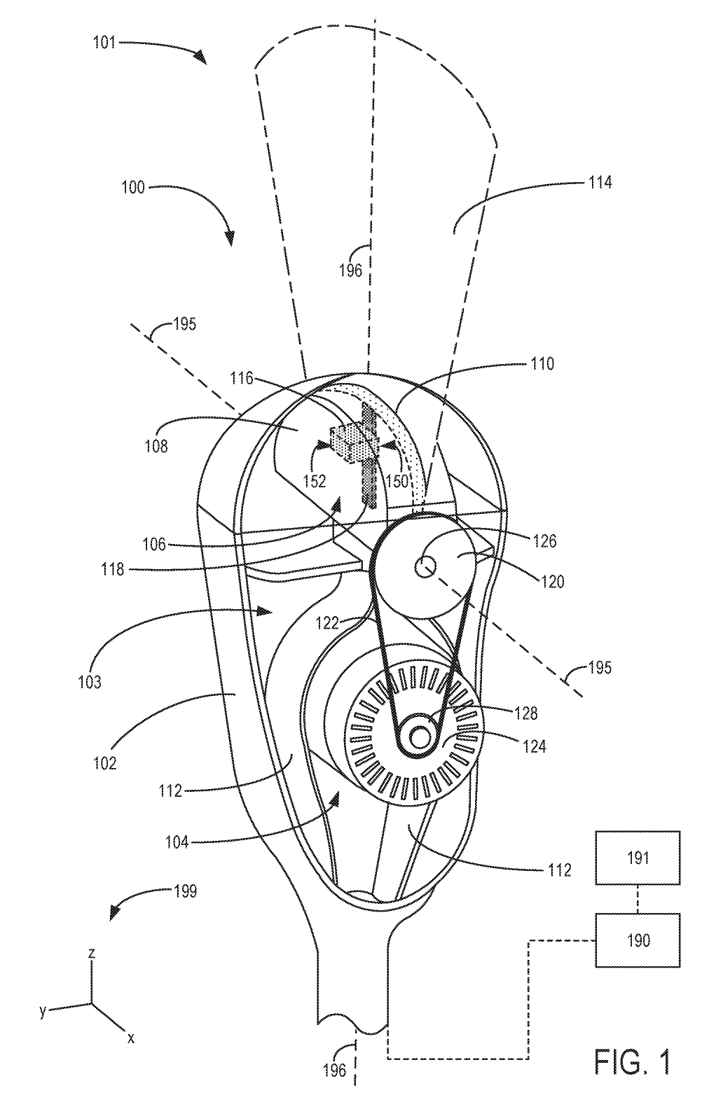

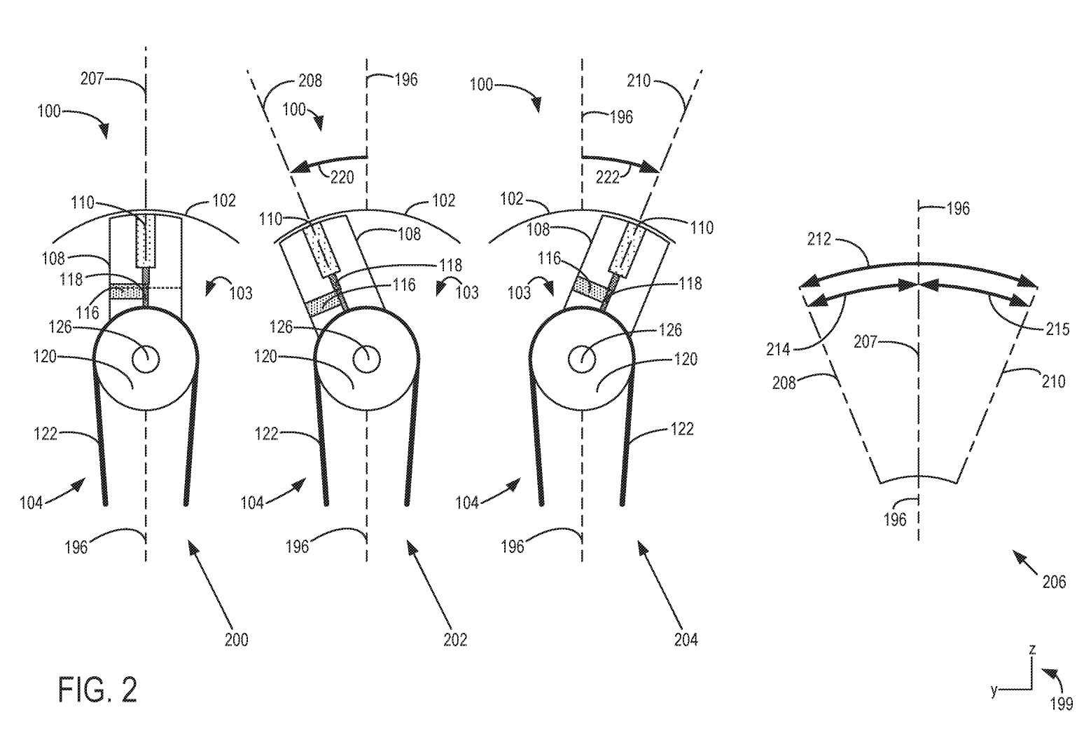

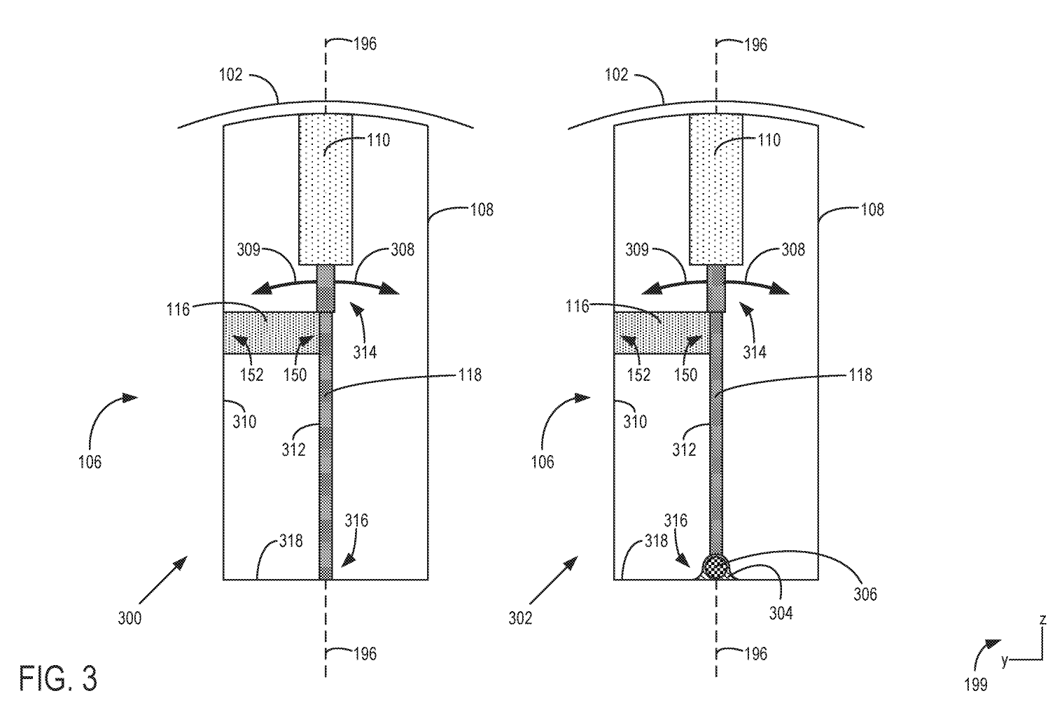

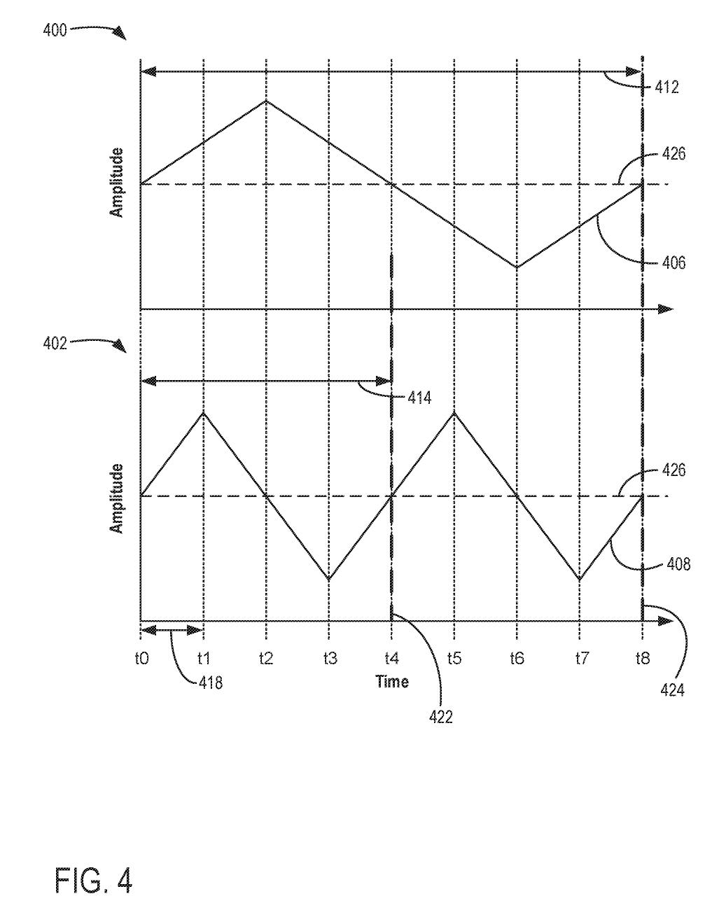

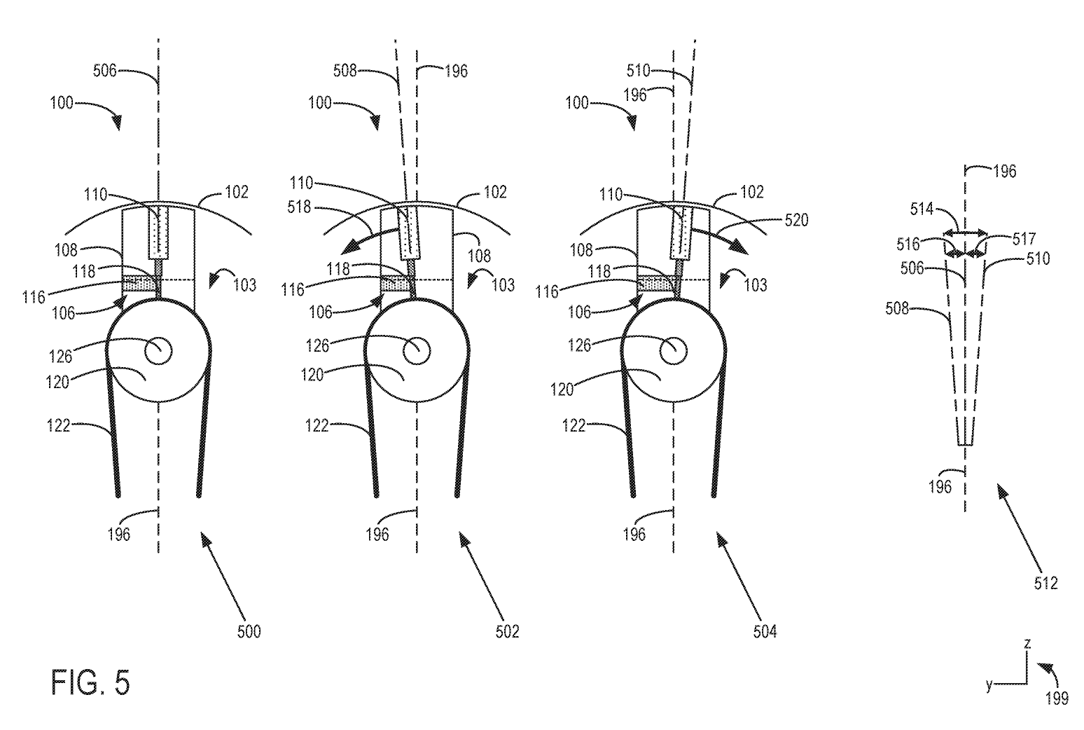

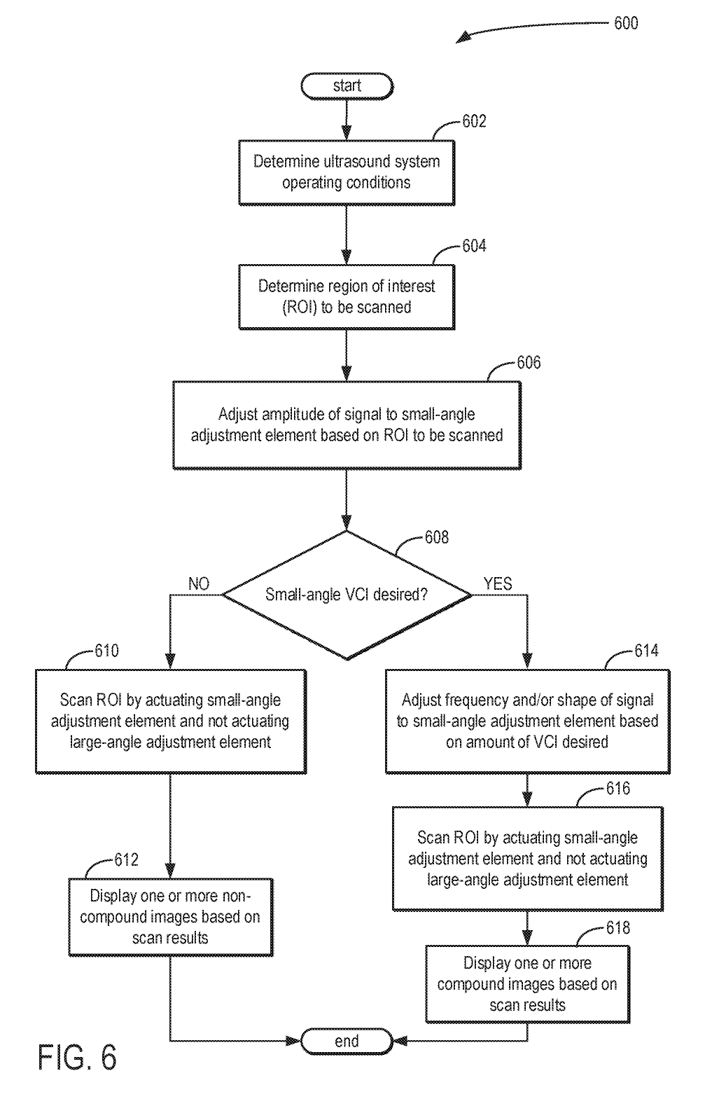

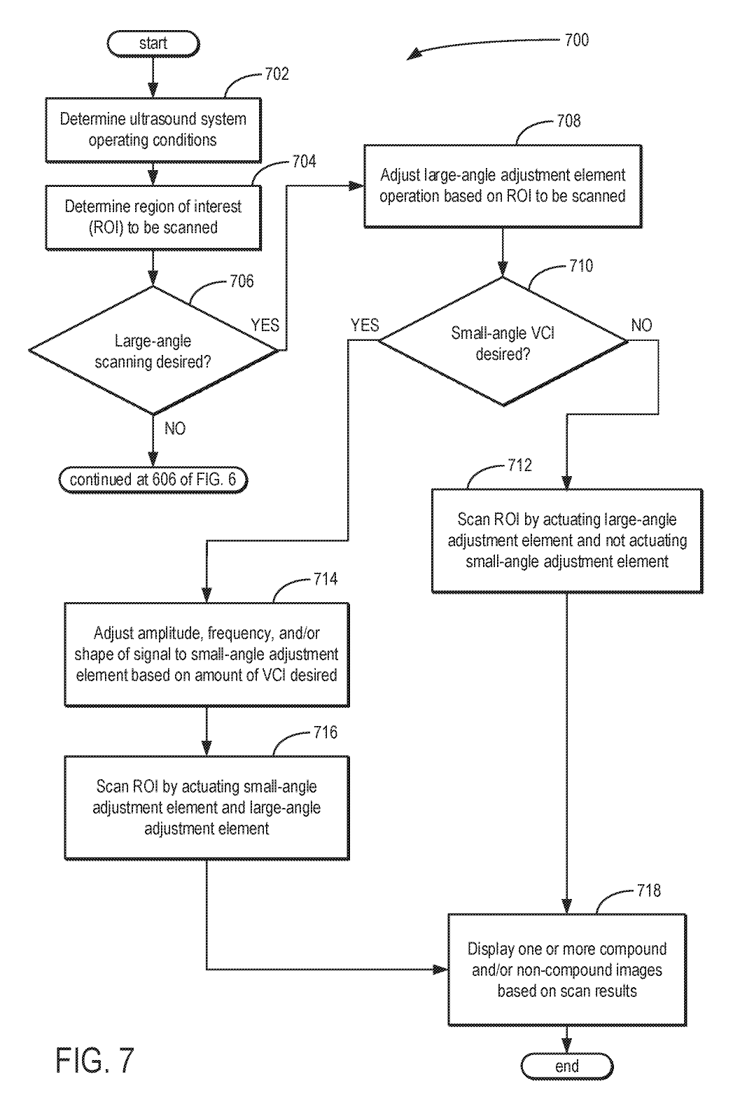

The following description relates to various embodiments of ultrasound systems. In particular, systems and methods are provided for an ultrasound probe including a large-angle adjustment element and a small-angle adjustment element. An example of an ultrasound probe that may be used to acquire images with small-angle scans and/or large-angle scans is provided in FIG. 1. In one example, the large-angle adjustment element may be a stepper motor used to rotate a sensor housing of the ultrasound probe to perform large-angle scans, as shown by FIG. 2. The small-angle adjustment element may be a piezoelectric element coupled to a pivotable support, as shown by FIG. 3. The piezoelectric element may be actuated by various signal waveforms, such as those shown by FIG. 4, to rotate a transducer array within the sensor housing to perform small-angle scans, as shown by FIG. 5. In one example, the small-angle adjustment element may be actuated to acquire images across a small angle as shown by the method of FIG. 6. In another example, the small-angle adjustment element and large-angle adjustment element may be actuated together to acquire enhanced images across a large angle as shown by the method of FIG. 7.

Various views of an ultrasound probe including a small-angle adjustment element and a large-angle adjustment element are shown by FIGS. 1-3 and FIG. 5. Reference axes 199 are included by each of FIGS. 1-3 and FIG. 5 for comparison of the views shown.

FIG. 1 shows an ultrasound system 101 including an ultrasound probe 100 electrically coupled with a workstation 190. The workstation 190 is configured to receive electrical signals from the ultrasound probe 100 and process the electrical signals into images to be displayed on display device 191 (e.g., a flat screen display). In one example, the display device may be a touch-sensitive display, also referred to as a touchscreen. An operator of the ultrasound system 101 may interact with the touchscreen to analyze the displayed image. In some examples, one or more additional user interface elements (e.g., mouse and keyboard) may be electrically coupled with the workstation 190 and configured such that an operator of the ultrasound system 101 may interact with (e.g., select, manipulate, etc.) images displayed by display device 191.

In one example, the workstation 190 is a computer including a microprocessor unit, input/output ports, an electronic storage medium (such as a non-transitory read only memory chip) for storing executable programs, instructions, and calibration values, random access memory, keep alive memory, and a data bus. The workstation 190 receives signals from the ultrasound probe 100 via cables 112 electrically coupled to a transducer array 110.

Ultrasound probe 100 includes a main housing 102 shown in cross-section by FIG. 2. A large-angle adjustment element 104 is included within an interior 103 of the ultrasound probe 100. In the examples shown by FIGS. 1-3 and FIG. 5, the large-angle adjustment element 104 includes a stepper motor 124 configured to drive a first pulley 120 via belt 122. In alternate embodiments, the large-angle adjustment element 104 may be a different type of actuator (e.g., a servomotor).

Stepper motor 124 includes a second pulley 128 rotatably coupled with the stepper motor 124. The ultrasound probe 100 may be coupled to an electrical power source in order to drive components of the ultrasound probe 100 (e.g., stepper motor 124, transducer array 110, etc.). In one example, the electrical power source may be the workstation 190, and the stepper motor 124 may be energized by the workstation 190 according to instructions stored in non-transitory memory of the workstation 190 (as described below with reference to FIGS. 6-7). Energization of the stepper motor 124 may cause second pulley 128 to rotate, thereby driving belt 122 to rotate first pulley 120. First pulley 120 is fixedly coupled with a sensor housing 108 within the main housing 102 by a fastener 126 (e.g., a bolt). In this configuration, as the first pulley 120 is rotated in a first direction by a first amount via the belt 122, the sensor housing 108 is also rotated in the first direction by the first amount. Said another way, the first pulley 120 rotates around rotational axis 195 when driven by the stepper motor 124 via belt 122, and the rotation of first pulley 120 results in a pivoting of the sensor housing 108 relative to the rotational axis 195 due to sensor housing 108 being directly coupled to first pulley 120 via fastener 126.

The transducer array 110 of the ultrasound probe 100 includes a plurality of decoupled electric and acoustic modules. The transducer array 110 is typically used to transmit ultrasonic or acoustic waves towards an object (not shown in FIG. 1). In response to transmitting the ultrasonic waves, the transducer array 110 may receive reflected or attenuated ultrasonic waves from the object. Further, these received ultrasonic waves are converted into electrical signals which are processed by the workstation 190 to obtain an ultrasonic image of the object. In one embodiment, the object may be a region of interest in a patient.

The transducer array 110 is coupled to the sensor housing 108 such that the transducer array 110 rotates (e.g., pivots) along with the sensor housing 108. In other words, by rotating the sensor housing 108 via the stepper motor 124 as described above, the transducer array 110 is also rotated. In the example shown by FIG. 1, the transducer array 110 is coupled to the sensor housing 108 via a shaft 118. In one example, shaft 118 extends in a direction perpendicular with the rotational axis 195 and provides a rigid connection between transducer array 110 and the sensor housing 108. Said another way, the shaft 118 may be directly coupled to the sensor housing 108 at an end opposite to an end coupled with the transducer array 110. In this configuration, when the sensor housing 108 is pivoted around the rotational axis 195 by a first amount of angle, the transducer array 110 is also pivoted around the rotational axis 195 by the first amount of angle.

By rotating the transducer array 110 via the stepper motor 124, an imaging plane 114 produced by the transducer array 110 is also rotated (e.g., pivoted). The imaging plane 114 shown by FIG. 1 represents a plane on which acoustic waves produced by the transducer array 110 travel when the transducer array 110 is energized by the workstation 190. The acoustic waves may travel through an object (e.g., a region of interest on a patient) and reflect off of structures (e.g., tissues, blood vessels, etc.) internal to the object. The reflected waves may then return to the transducer array 110 along the imaging plane 114, with the mechanical energy of the waves being converted into electrical signals by the transducer array 110. The transducer array 110 then transmits the electrical signals to the workstation 190 via the cables 112, where the workstation 190 processes the electrical signals to form an image on display device 191. The acoustic waves transmitted along imaging plane 114 thereby characterize the image processed by the workstation 190 and shown on display device 191. By rotating the imaging plane 114 via the stepper motor 124 as described above, the ultrasound probe 100 may gather images from a variety of angles without adjustment of a position of the ultrasound probe 100 (e.g., a position of the main housing 102) by an operator of the ultrasound system 101.

The shaft 118 is shown as part of a small-angle adjustment element 106. The small-angle adjustment element 106 includes a piezoelectric actuator 116 directly coupled to (e.g., in face-sharing contact with) the shaft 118. Piezoelectric actuator 116 is also electrically coupled with the electrical power source (e.g., workstation 190) by one or more conductive wires within cable 112 (for example). The piezoelectric actuator 116 may be directly coupled with the shaft 118 at a first end 150, and may be directly coupled with the sensor housing 108 at a second end 152. Piezoelectric actuator 116 is configured to expand or contract in size in response to electrical signals transmitted to the piezoelectric actuator 116 by the workstation 190.

For example, the workstation 190 may transmit an electrical signal to the piezoelectric actuator 116 resulting in a positive-valued voltage across the piezoelectric actuator 116. As a result of the positive-valued voltage, the piezoelectric actuator 116 expands in a direction perpendicular to the shaft 118. In other words, a length of the piezoelectric actuator 116 extending from the first end 150 coupled with the shaft 118 to the second end 152 coupled with the sensor housing 108 is increased by the positive-valued voltage. The piezoelectric actuator 116 thereby exerts a pressing force against the shaft 118 to pivot the shaft 118 and transducer array 110 relative to the sensor housing 108 in a first direction (as shown by FIG. 5 and described further below).

In another example, an electrical signal transmitted to the piezoelectric actuator 116 by the workstation 190 may result in a negative-valued voltage across the piezoelectric actuator 116. As a result of the negative-valued voltage, a length of the piezoelectric actuator 116 extending between the first end 150 and second end 152 is decreased. The piezoelectric actuator 116 thereby exerts a pulling force against the shaft 118 to pivot the shaft 118 and transducer array 110 relative to the sensor housing 108 in a second direction opposite to the first direction (as shown by FIG. 5 and described below).

By configuring the ultrasound probe 100 according to the examples described above, the stepper motor 124 may rotate (e.g., pivot) the sensor housing 108 (and therefore, the transducer array 110 and imaging plane 114) relative to the main housing 102 in order to perform relatively large-angle scans across a region of interest on a patient (for example). Additionally, the piezoelectric actuator 116 may be energized to pivot the transducer array 110 and imaging plane 114 relative to the sensor housing 108 in order to perform relatively small-angle scans across the region of interest. In some examples, the stepper motor 124 may be energized while the piezoelectric actuator 116 is not energized (e.g., in order to perform a large volumetric scan). In other examples, the piezoelectric actuator 116 may be energized while the stepper motor 124 is not energized (e.g., in order to enhance a scan using volume compound imaging). In yet other examples, both of the piezoelectric actuator 116 and stepper motor 124 may be energized (e.g., in order to perform a volumetric scan with volume compound imaging). Example scanning methods are described further below with reference to FIGS. 6-7.

FIG. 2 shows views (e.g., first view 200, second view 202, and third view 204) of the sensor housing 108 in various positions relative to the main housing 102. Each of the first view 200, second view 202, and third view 204 show a portion of the ultrasound probe 100 in partial cross-section in order to illustrate a movement of the sensor housing 108 relative to the main housing 102. As a result, some components included by the ultrasound probe 100 and shown by FIG. 1 are not shown by FIG. 2. FIG. 2 additionally includes a fourth view 206, with the fourth view 206 showing a positioning of a first imaging plane 207, a second imaging plane 208, and a third imaging plane 210 relative to each other. The first imaging plane 207 is produced by the transducer array 110 when the sensor housing 108 is in the position shown by the first view 200, the second imaging plane 208 is produced by the transducer array 110 when the sensor housing 108 is in the position shown by the second view 202, and the third imaging plane 210 is produced by the transducer array 110 when the sensor housing 108 is in the position shown by the third view 204, as described below.

First view 200 shows an initial position of the sensor housing 108 in which the sensor housing 108 is not rotated relative to the main housing 102 (e.g., first imaging plane 207 produced by the transducer array 110 is aligned with the central axis 196). The initial position of the sensor housing 108 shown by first view 200 may be referred to herein as a non-rotated position and/or reference position of the sensor housing 108.

Second view 202 shows the sensor housing 108 rotated in a first direction 220 relative to the main housing 102, such that the second imaging plane 208 is also rotated in the first direction 220 relative to the first imaging plane 207. In other words, the second imaging plane 208 is at a first angle 214 relative to the central axis 196 and first imaging plane 207 as shown by fourth view 206 and described below. In one example, the sensor housing 108 may be rotated in the first direction 220 from the position shown by first view 200 to the position shown by second view 202 by the large-angle adjustment element 104 (e.g., via energization of the stepper motor 124 by the workstation 190, both of which are shown by FIG. 1 and described above).

Third view 204 shows the sensor housing 108 rotated in a second direction 222 relative to the main housing 102, such that the third imaging plane 210 is also rotated in the second direction 222 relative to the first imaging plane 207. In other words, the third imaging plane 210 is at a second angle 215 relative to the central axis 196 and first imaging plane 207. In one example, the sensor housing 108 may be rotated in the second direction 222 from the position shown by first view 200 to the position shown by third view 204 by the large-angle adjustment element 104 (e.g., via energization of the stepper motor 124 by the workstation 190, both of which are shown by FIG. 1 and described above).

Fourth view 206 shows a relative arrangement of the first imaging plane 207, second imaging plane 208, third imaging plane 210, and central axis 196. As described above, second imaging plane 208 is angled relative to the first imaging plane 207 by a first angle 214, and third imaging plane 210 is angled relative to the first imaging plane 207 by a second angle 215. In one example, the first angle 214 and second angle 215 are a same amount of angle extending in opposite directions (e.g., first direction 220 and second direction 222, respectively) from the central axis 196.

During operation of the ultrasound probe 100, the large-angle adjustment element 104 may be actuated by the workstation 190 (described below with reference to FIG. 7) in order to perform a large-angle scan of a region of interest of an object (e.g., a patient). The large-angle scan may include images gathered by the ultrasound probe 100 along each of the first imaging plane 207, second imaging plane 208, and third imaging plane 210. In other words, the large-angle scan may span an angle 212, with the angle 212 extending between the second imaging plane 208 and third imaging plane 210.

It should be noted that the large-angle scan spanning the angle 212 shown by FIG. 2 is one example of a large-angle scan. In other examples, the large-angle scan may span a different amount of angle than the angle 212 and may include a plurality of imaging planes different than those shown by FIG. 2. In one example, the angle 212 may be 45 degrees, and the first angle 214 and second angle 215 may each be 22.5 degrees. In another example the angle 212 may be 50 degrees, and the number of imaging planes may be greater than 3. Additional examples of angle and number of imaging planes are also possible. However, in each example, the angle 212 of the large-angle scan is greater than an angle of a small-angle scan produced by actuation of the small-angle adjustment element 106 (shown by FIG. 1), as described below with reference to FIGS. 3-5.

FIG. 3 shows an enlarged view of the small-angle adjustment element 106 in a first view 300, with an alternate embodiment of the small-angle adjustment element 106 shown by second view 302. The small-angle adjustment element 106 includes the piezoelectric actuator 116 coupled between the sensor housing 108 and the shaft 118 as described above with reference to FIG. 1. In particular, the second end 152 of the piezoelectric actuator 116 is coupled to an interior wall 310 of the sensor housing 108, and the first end 150 of the piezoelectric actuator 116 is coupled to a first surface 312 of the shaft 118, with the first surface 312 and interior wall 310 extending in a direction approximately parallel with each other when the piezoelectric actuator 116 is in a de-energized condition (e.g., not energized by workstation 190 shown by FIG. 1). In this configuration, the piezoelectric actuator 116 extends between the interior wall 310 and the first surface 312 in a direction perpendicular with the interior wall 310.

In the example shown by first view 300, a first end 314 of the shaft 118 is coupled to the transducer array 110 and a second end 316 of the shaft 118 is coupled directly to a lower wall 318 of the sensor housing 108. The lower wall 318 is joined with the interior wall 310 and extends in a direction perpendicular with the interior wall 310. The second end 316 of the shaft 118 is permanently joined with (e.g., fused or welded to) the lower wall 318. The first end 314 coupled with the transducer array 110 is pivotable relative to the second end 316 in a first direction 308 in response to a pressing force against the second end 316, and the first end 314 is pivotable in a second direction 309 (opposite to the first direction 308) in response to a pulling force against the second end 316. Both of the pressing force and pulling force may be in directions approximately perpendicular with the direction of extension of shaft 118.

In a second example shown by second view 302, the second end 316 of the shaft 118 is instead coupled to a bearing 306 supported within a bearing housing 304. The bearing housing 304 is coupled to the lower wall 318, and the bearing 306 is shaped to rotate within the bearing housing 304 in response to a pivoting of the shaft 118. For example, a pressing force may press against the first end 314 in order to pivot the shaft 118 relative to the bearing housing 304 in the first direction 308. In another example, a pulling force may pull against the first end 314 to pivot the shaft 118 relative to the bearing housing 304 in the second direction 309.

In one example (as described above and with reference to FIG. 1), the pressing force and pulling force may result from an expansion or contraction of the piezoelectric actuator 116 when the piezoelectric actuator 116 is energized by the workstation 190 (shown by FIG. 1). For example, the workstation 190 may transmit an electrical signal to the piezoelectric actuator 116 to apply a negative-valued voltage across the piezoelectric actuator 116, thereby resulting in the first end 150 and second end 152 of the piezoelectric actuator 116 moving toward each other (e.g., shortening the piezoelectric actuator 116). In another example, the workstation 190 may transmit an electrical signal to the piezoelectric actuator 116 to apply a positive-valued voltage across the piezoelectric actuator 116, thereby resulting in the first end 150 and second end 152 of the piezoelectric actuator moving away from each other (e.g., lengthening the piezoelectric actuator 116). Example signals that may be transmitted to the piezoelectric actuator 116 are shown by FIG. 4 and described below.

FIG. 4 shows two example electrical signals (e.g., pulses) that may be transmitted to piezoelectric actuator 116 (shown by FIGS. 1-3 and FIG. 5). Specifically, first plot 400 shows a first signal 406 with a first wavelength 412 and second plot 402 shows a second signal 408 with a second wavelength 414. In the example shown by FIG. 4, second wavelength 414 has a length that is one-half an amount of a length of first wavelength 412. Although the first signal 406 and second signal 408 are each shown as triangular waves, other wave shapes (e.g., sine waves, square waves, sawtooth waves, etc.) are possible. A threshold amplitude 426 is shown by each of the first plot 400 and second plot 402. In one example, when an amplitude of a signal (e.g., first signal 406 and second signal 408) is above the threshold amplitude 426, the signal may result in a positive-valued voltage across the piezoelectric actuator, and when the amplitude of the signal is below the threshold amplitude 426, the signal may result in a negative-valued voltage across the piezoelectric actuator.

Each of the times t0 through t8 shown by FIG. 4 correspond to times at which the transducer array 110 of the ultrasound probe 100 (shown by FIGS. 1-3 and FIG. 5) may transmit electrical signals to the workstation 190 (e.g., times at which echoes from transmitted acoustic waves may be received). The workstation 190 may process the electrical signals to form an image (e.g., a frame) associated with each of the times t0 through t8. For example, at time t0, the transducer array may receive echoes from a previously transmitted acoustic wave (e.g., prior to time t0) within an object (e.g., a patient). The mechanical energy of the echoes is converted into electrical energy by the transducer array, and the transducer array transmits the electrical energy to the workstation in the form of an electrical signal. The workstation then processes the electrical signal to form an image which may be displayed on a display device (e.g., display device 191 shown by FIG. 1).

In the example shown by FIG. 4, an amount of time 418 between time t0 and time t1 is a same amount of time as times between each adjacent time of t0 through t8. In other words, the amount of time 418 between time t0 and time t1 is a same amount of time as an amount of time between t1 and t2, between t2 and t3, between t3 and t4, etc. In one example, the amount of time 418 may correspond to an amount of time between transmission of acoustic waves from the transducer array and reception of echoes of the acoustic waves by the transducer array. An example of ultrasound probe operation in response to first signal 406 being transmitted to the ultrasound probe via the workstation is described below.

As shown by first plot 400, the first wavelength 412 of the first signal 406 extends between time t0 and time t8. Between time t0 and time t2, the amplitude of the first signal 406 is above the threshold amplitude 426 and is increasing. The amplitude between time t0 and time t2 results in a positive-valued voltage across the piezoelectric actuator, thereby causing the piezoelectric actuator to increasingly expand. The expansion of the piezoelectric actuator results in a transducer array being rotated in a first direction relative to the sensor housing (e.g., as described above with reference to transducer array 110 and sensor housing 108 shown by FIGS. 1-3, with the transducer array 110 rotated in the first direction 308 shown by FIG. 3). At time t2 the amplitude of the first signal 406 peaks. As a result, the piezoelectric actuator does not expand further and the transducer array does not rotate further in the first direction.

Between time t2 and time t4, the amplitude of the first signal 406 is above the threshold amplitude 426 and is decreasing. As a result, a magnitude of the positive-valued voltage across the piezoelectric actuator decreases, and the piezoelectric actuator begins to contract relative to its fully expanded condition at time t2. The contraction of the piezoelectric actuator causes the transducer array 110 to rotate in a second direction (e.g., second direction 309 shown by FIG. 3) opposite to the first direction.

At time t4 the amplitude of the first signal 406 decreases below the threshold amplitude 426, and between time t4 and t6 the amplitude of the first signal 406 continues to decrease. The amplitude of the first signal 406 being below the threshold amplitude 426 results in a negative-valued voltage across the piezoelectric actuator, thereby causing the piezoelectric actuator to increasingly contract. The contraction of the piezoelectric actuator results in a continued rotation of the transducer array in the second direction as described above. At time t6 the amplitude of the first signal 406 is at a peak negative value. As a result, the piezoelectric actuator does not contract further and the transducer array does not rotate further in the second direction.

Between time t6 and time t8, the amplitude of the first signal 406 is below the threshold amplitude 426 and is increasing. As a result, the negative-valued voltage across the piezoelectric actuator becomes increasingly less negative-valued, and the piezoelectric actuator expands relative to its fully contracted condition (e.g., the condition of the piezoelectric actuator at time t6). The expansion of the piezoelectric actuator causes the transducer array to rotate in the first direction.

By rotating the transducer array in the first direction and second direction in response to the amplitude of the first signal 406 as described above, an angle of the transducer array relative to the sensor housing may be different at one or more or each of the times t0 through t8. For example, the transducer array may be at a first angle relative to the sensor housing at time t4, but may be angled toward the first direction (e.g., first direction 308 shown by FIG. 3) at time t3, and may be angled toward the second direction (e.g., second direction 309 shown by FIG. 3) at time t5. Imaging planes produced by the transducer array at each of the times t0 through t8 may be angled similarly to the example described above. With reference to the example described above, at time t4 the imaging plane produced by the transducer array is at the first angle, while at time t3 the imaging plane is angled toward the first direction relative to the first angle, and at time t5 the imaging plane is angled toward the second direction relative to the first angle. An example of a relative positioning of imaging planes is shown by FIG. 5 and described below.

In this way, as the transducer array receives echoes at each of the times t0 through t8 along the imaging planes as described above, the ultrasound probe transmits a series of electrical signals to the workstation and the workstation processes the electrical signals to form a plurality of images. Each image of the plurality of images is associated with one of the times t0 through t8. For example, the workstation may associate a first image of the plurality of images with echoes received at time t0, a second image of the plurality of images with echoes received at time t1, a third image of the plurality of images with echoes received at time t2, etc. In one example, the images may be associated by the workstation such that (for example) the first image associated with time t0 by the workstation is an image processed from echoes received along an imaging plane of the transducer array at time t0. In some examples (as described below with reference to FIGS. 6-7) the workstation may compare at least two images of the plurality of images to each other in order to form a volume compound image for display on a display device (e.g., display device 191 shown by FIG. 1). The volume compound image may have an increased clarity and/or contrast relative to the at least two images of the plurality of images.

Second signal 408 is shown as an example of a signal with an increased frequency relative to first signal 406. Because the second wavelength 414 of second signal 408 is one-half the amount of length of first wavelength 412, a rotation speed of the transducer array due to the expansion and contraction of the piezoelectric actuator (as described above with reference to the first signal 406) is increased relative to the example described above. As a result, the transducer array may move over a region of interest more quickly when energized via the second signal 408 compared to the first signal 406. However, due to the amount of time 418 between the reception of echoes by the ultrasound probe as described above, a number of images processed by the workstation for each wavelength of second signal 408 is reduced relative to a number of images processed by the workstation for each wavelength of first signal 406.

For example, one wavelength of the first signal 406 (e.g., first wavelength 412) spans the time between time t0 and time t8 (as indicated by dotted line 424 signifying an end of first wavelength 412). As such, for the first wavelength of the first signal 406, eight images (one for each of times t0 through t7) may be processed by the workstation, with the time t8 corresponding to an end of first wavelength 412 and a beginning of a next wavelength of first signal 406. However, one wavelength of the second signal 408 (e.g., second wavelength 414) spans the time between time t0 and time t4, for example (as indicated by dotted line 422 signifying an end of second wavelength 414). As a result, four images (one for each of times t0 through t3) are associated with the second wavelength 414 of the second signal 408, with the time t4 corresponding to an end of second wavelength 414 and a beginning of a next wavelength of second signal 408. In one example, the number of images associated with each wavelength may contribute to a clarity and/or contrast of a volume compound image produced by the workstation for each wavelength (e.g., each scan of the region of interest via rotation of the transducer array). In other words, decreasing a wavelength of a signal transmitted to the piezoelectric actuator may increase a rotational speed of the transducer array in order to increase a scanning speed of the region of interest by the ultrasound probe, but may reduce a number of images gathered by the ultrasound probe during each scan. In one example, an operator of the ultrasound probe may interact with the workstation in order to set the wavelength of the electrical signal transmitted to the piezoelectric actuator in order to increase the scanning speed or increase the number of images gathered per scan. An example of scan is shown by FIG. 5 and described below.

FIG. 5 shows views (e.g., first view 500, second view 502, and third view 504) of the transducer array 110 in various positions relative to the sensor housing 108. Each of the first view 500, second view 502, and third view 504 show a portion of the ultrasound probe 100 in partial cross-section in order to illustrate a rotation (e.g., pivoting) of the transducer array 110 relative to the sensor housing 108. As a result, some components included by the ultrasound probe 100 and shown by FIG. 1 are not shown by FIG. 5. FIG. 5 additionally includes a fourth view 512, with the fourth view 512 showing a positioning of a first imaging plane 506, a second imaging plane 508, and a third imaging plane 510 relative to each other. The first imaging plane 506 is produced by the transducer array 110 when the transducer array 110 is in the position shown by the first view 500, the second imaging plane 508 is produced by the transducer array 110 when the transducer array 110 is in the position shown by the second view 502, and the third imaging plane 510 is produced by the transducer array 110 when the transducer array 110 is in the position shown by the third view 504, as described below.

First view 500 shows an initial position of the transducer array 110 in which the transducer array 110 is not rotated relative to the sensor housing 108. The initial position of the transducer array 110 shown by first view 500 may be referred to herein as a non-rotated position and/or reference position of the transducer array 110. As described above, the small-angle adjustment element 106 may be energized by the workstation 190 (shown by FIG. 1) in order to rotate the transducer array 110 relative to the sensor housing 108. In the example described above with reference to FIGS. 1-4, the piezoelectric actuator 116 of the small-angle adjustment element 106 may be energized by an electrical signal such as those described with reference to FIG. 4 in order to rotate transducer array 110 relative to sensor housing 108.

Second view 502 shows the transducer array 110 rotated in a first direction 518 relative to the sensor housing 108, such that the second imaging plane 508 is also rotated in the first direction 518 relative to the first imaging plane 506. In other words, the second imaging plane 508 is at a first angle 516 relative to the first imaging plane 506 as shown by fourth view 512 and described below. In one example, the transducer array 110 may be rotated in the first direction 518 from the position shown by first view 500 to the position shown by second view 502 by the small-angle adjustment element 106 (e.g., via energization of the piezoelectric actuator 116 by the workstation 190, both of which are shown by FIG. 1 and described above).

Third view 504 shows the transducer array 110 rotated in a second direction 520 relative to the sensor housing 108, such that the third imaging plane 510 is also rotated in the second direction 520 relative to the first imaging plane 506. In other words, the third imaging plane 510 is at a second angle 517 relative to the first imaging plane 506. In one example, the transducer array 110 may be rotated in the second direction 520 from the position shown by first view 500 to the position shown by third view 504 by the small-angle adjustment element 106 (e.g., via energization of the piezoelectric actuator 116 by the workstation 190, both of which are shown by FIG. 1 and described above).

Fourth view 512 shows a relative arrangement of the first imaging plane 506, second imaging plane 508, third imaging plane 510, and central axis 196. As described above, second imaging plane 508 is angled relative to the first imaging plane 506 by a first angle 516, and third imaging plane 510 is angled relative to the first imaging plane 508 by a second angle 517. In one example, the first angle 516 and second angle 517 are a same amount of angle extending in opposite directions (e.g., first direction 518 and second direction 520, respectively) from the first imaging plane 506.

During operation of the ultrasound probe 100, the small-angle adjustment element 106 may be actuated by the workstation 190 (described below with reference to FIGS. 6-7) in order to perform a small-angle scan of a region of interest of an object (e.g., a patient). In one example, the small-angle scan may include images gathered by the ultrasound probe 100 along each of the first imaging plane 506, second imaging plane 508, and third imaging plane 510. In other words, the small-angle scan may span an angle 514, with the angle 514 extending between the second imaging plane 506 and third imaging plane 510.

It should be noted that the small-angle scan spanning the angle 514 shown by FIG. 5 is one example of a small-angle scan. In other examples, the small-angle scan may span a different amount of angle than the angle 514 and may include a plurality of imaging planes different than those shown by FIG. 5. In one example, the angle 514 may be 5 degrees, and the first angle 516 and second angle 517 may each be 2.5 degrees. In another example the angle 514 may be 7 degrees, and the number of imaging planes may be greater than 3. Additional examples of angle and number of imaging planes are also possible. However, in each example, the angle 514 of the small-angle scan is much smaller (e.g., 5 degrees) relative to an angle of a large-angle scan (e.g., 45 degrees) produced by actuation of the large-angle adjustment element 104 (shown by FIGS. 1-2), as described above with reference to FIG. 2.

FIG. 6 shows an example operation of an ultrasound probe including a small-angle adjustment element, such as the ultrasound probe described in the examples above with reference to FIGS. 1-3 and FIG. 5. The small-angle adjustment element may be configured to receive electrical signals from the workstation such as those shown by FIG. 4 and described above.

Instructions for carrying out method 600 shown by FIG. 6, method 700 shown by FIG. 7, and the rest of the methods included herein may be executed by a controller (e.g., workstation 190 shown by FIG. 1) based on instructions stored on a memory of the controller and in conjunction with signals received from the ultrasound probe, such as the transducer array described above with reference to FIGS. 1-5. The controller may employ actuators of the ultrasound probe to adjust ultrasound probe operation, according to the methods described below.

At 602, the method includes determining ultrasound system operating conditions. In one example, determining ultrasound system operating conditions may include determining a position of the transducer array relative to the sensor housing, determining a position of the sensor housing relative to the main housing, determining electrical signals transmitted to the small-angle adjustment element, determining images stored in workstation memory and/or displayed on the display device, etc.

The method continues from 602 to 604 where the method includes determining a region of interest (ROI) to be scanned. In one example, an ROI may be a region of interest on a body of a patient (e.g., an abdomen of a patient). An operator of the ultrasound system (e.g., an ultrasound technician) may determine the ROI in order to position the ultrasound probe of the ultrasound system such that one or more scans of the region may be performed. In the example above, the ultrasound probe may be positioned against the abdomen in order to scan a region of the abdomen.

The method continues from 604 to 606 where the method includes adjusting an amplitude of a signal transmitted to the small-angle adjustment element based on the region of interest to be scanned. In one example, the amplitude of the signal may be increased in order to increase a rotation amount of the transducer array relative to the sensor housing (as described above with reference to FIG. 4). By increasing the amplitude of the signal and the rotation amount of the sensor array, a larger amount of the region of interest may be scanned within a single scan. In another example, the amplitude of the signal may be decreased in order to decrease the rotation amount of the transducer array relative to the sensor housing. By decreasing the amplitude of the signal and the rotation amount of the sensor array, a narrower region may be scanned.

The method continues from 606 to 608 where the method includes determining whether small-angle volume compound imaging (VCI) is desired. For example, the operator of the system may determine that small-angle VCI is desired in order to increase a clarity and/or contrast of images of the region of interest. The workstation may compare and/or combine a first plurality of images formed during a single scan in order to produce one or more compound images, with an amount of the one or more compound images being less than an amount of the first plurality of images. The one or more compound images may have an increased clarity and/or contrast (e.g., reduction of acoustical artifacts) relative to the first plurality of images.

In another example, the operator of the system may determine that small-angle VCI is not desired. For example, the region of interest may be a region in which images produced by the ultrasound system typically do not include a high amount of acoustical artifacts, image noise, etc. As a result, the operator may determine that producing compound images via small-angle VCI is less desirable than acquiring non-compound images (e.g., images without VCI) at an increased rate.

If small-angle VCI is not desired at 608, the method continues to 610 where the method includes scanning the ROI by actuating the small-angle adjustment element. For example, the small-angle adjustment element may be actuated via an electrical signal transmitted from the workstation to a piezoelectric element included by the small-angle adjustment element (e.g., piezoelectric actuator 116 described above with reference to FIG. 1, FIG. 3, etc.). In one example, the electrical signal may be similar to the signals shown by FIG. 4 and described above. In other words, the transducer array is rotated by actuation of the piezoelectric element (as described above with reference to FIGS. 3-5). In this way, the ultrasound probe may perform a small-angle scan via energization of the piezoelectric element, as described above with reference to FIGS. 4-5.

The method continues from 610 to 612 where the method includes displaying one or more non-compound images based on scan results. In some examples, images acquired during the small-angle scan described above with reference to 610 may be displayed by a display device electrically coupled with the workstation (e.g., display device 191 shown by FIG. 1). For example, the workstation and display device may be configured to display each of the images acquired during the small-angle scan in an order in which they were acquired. In another example, the workstation and display device may be configured to display each of the images as a side-by-side comparison. In yet another example, the workstation and display device may be configured to display images associated with imaging planes chosen by the operator of the ultrasound system. The operator may select between display modes (e.g., the workstation and display device configurations described above) according to operator preference, and other display modes may be possible.

Returning to 608, if the small-angle VCI is desired at 608, the method continues to 614 where the method includes adjusting a frequency and/or a shape of the signal to the small-angle adjustment element based on an amount of VCI desired. For example, as described above with reference to FIG. 4, a frequency of the electrical signal transmitted to the piezoelectric element may be decreased in order to increase a number of images acquired during a single small-angle scan. By increasing the number of images acquired, the workstation may compare and/or combine an increased number of images with each other in order to produce a compound image via VCI. The increased number of images thereby decreases an amount of acoustical artifacts, image noise, etc. included by the compound image.

In some examples, a shape of the waveform of the electric signal transmitted to the piezoelectric actuator may be adjusted in order to increase an amount of VCI along imaging planes selected by the operator of the ultrasound system. For example, an asymmetric waveform may be selected in order to acquire an increased number of images via imaging planes angled toward a first direction (e.g., first direction 518 shown by FIG. 5) compared to a decreased number of images via imaging planes angled toward a second direction (e.g., second direction 520 shown by FIG. 5). When the workstation compares and/or combines the images to form one or more compound images as described below, an averaging of the images may be weighted toward one or more regions of the scan in which a higher amount of VCI is desired. In other words, the operator of the ultrasound system may select a waveform configured to acquire a higher number of images in a first portion of the region of interest that typically has a high incidence of acoustical artifacts and/or image noise and a lower number of images in a second portion of the region of interest that typically has a low incidence of acoustical artifacts and/or image noise. In this way, a clarity and/or contrast of a compound image produced by the ultrasound system may be increased.

The method continues from 614 to 616 where the method includes scanning the ROI by actuating the small-angle adjustment element. As described above with reference to FIGS. 4-5, the small-angle adjustment element may be actuated via an electrical signal transmitted from the workstation to the piezoelectric element included by the small-angle adjustment element (e.g., piezoelectric actuator 116 described above with reference to FIG. 1, FIG. 3, etc.). In one example, the electrical signal may be similar to the signals shown by FIG. 4 and described above. In other examples, the electrical signal may have a different type of waveform, such as the asymmetrical waveform described above. The transducer array may be rotated by actuation of the piezoelectric element as described above with reference to FIGS. 3-5. In this way, the ultrasound probe may perform a small-angle scan via energization of the piezoelectric element, as described above with reference to FIGS. 4-5. At least a portion of the images acquired during the small-angle scan may then be compared and/or combined by the workstation as described below with reference to 618.

The method continues from 616 to 618 where the method includes displaying a compound image based on scan results. Scan results include the plurality of images acquired during the small-angle scan. In some examples, at least one compound image resulting from the small-angle scan described above with reference to 616 may be displayed by the display device electrically coupled with the workstation (e.g., display device 191 shown by FIG. 1) at 618. For example, the workstation and display device may be configured to display a compound image formed from one or more of the images acquired during the small-angle scan described above with reference to 616. In other words, the workstation may compare one or more images from the small-angle scan to each other and apply an averaging algorithm (e.g., noise reduction algorithm) to combine the images into at least one compound image with reduced acoustical artifacts/aberrations. The at least one compound image may then be displayed by the display device. In some examples, each of the images acquired during the small-angle scan may be combined into a single compound image, and the single compound image may be displayed by the display device. In other examples, a first amount of images from the small-angle scan may be combined into a first compound image, and a second amount of images from the small-angle scan may be combined into a second compound image, and both of the first compound image and second compound image may be displayed by the display device. For example, the workstation and display device may be configured to display multiple compound images associated with different imaging planes chosen by the operator of the ultrasound system.

FIG. 7 shows another example operation of an ultrasound probe including a small-angle adjustment element and a large-angle adjustment element, such as the ultrasound probe described in the examples above with reference to FIGS. 1-3 and FIG. 5. The small-angle adjustment element may be configured to receive electrical signals from the workstation such as those shown by FIG. 4 and described above.

At 702, the method includes determining ultrasound probe operating conditions. As described above with reference to 602 of method 600 shown by FIG. 6, determining ultrasound system operating conditions may include determining a position of the transducer array relative to the sensor housing, determining a position of the sensor housing relative to the main housing, determining electrical signals transmitted to the small-angle adjustment element and/or large-angle adjustment element, determining images stored in workstation memory and/or displayed on the display device, etc.

The method continues from 702 to 704 where the method includes determining a region of interest (ROI) to be scanned. As described above with reference to 604 of method 600 shown by FIG. 6, in one example an ROI may be a region of interest on a body of a patient (e.g., an abdomen of a patient). An operator of the ultrasound system (e.g., an ultrasound technician) may determine the ROI in order to position the ultrasound probe of the ultrasound system such that one or more scans of the region may be performed. In the example above, the ultrasound probe may be positioned against the abdomen in order to scan a region of the abdomen.

The method continues from 704 to 706 where the method includes determining whether large-angle scanning is desired. For example, the operator of the ultrasound system may determine that large-angle scanning is desired in order to acquire images of an object to be scanned (e.g., a patient) over an increased volume (e.g., an increased amount of angle relative to a central axis of the ultrasound probe, as described above with reference to FIG. 2). In one example, the operator may desire a scanning angle of 45 degrees, as shown by FIG. 2 and described above, in order to locate a particular feature (e.g., a muscle, organ, etc.) within the region of interest.

If large-angle volumetric scanning is not desired at 706, the method continues to 606 of method 600 shown by FIG. 6 and described above, in order to perform a small-angle volumetric scanning.

If large-angle volumetric scanning is desired at 706, the method continues to 708 where the method includes adjusting large-angle adjustment element operation based on the ROI to be scanned. In one example, adjusting large-angle adjustment element operation may include adjusting a number and/or polarity of electrical pulses transmitted to a stepper motor (e.g., stepper motor 124 shown by FIG. 1 and described above) during a single scan. For example, the stepper motor may be configured to rotate a pulley (e.g., second pulley 128 shown by FIG. 1) by one increment (e.g., one “step”) in response to an electrical pulse transmitted to the stepper motor from the workstation. The polarity of the electrical pulse may result in a positive-valued current or a negative-valued current through terminals of the stepper motor, with the positive-valued current resulting in the stepper motor rotating the pulley by one step in a first direction, and the negative-valued current resulting in the stepper motor rotating the pulley by one step in a second direction opposite to the first direction.

As described above with reference to FIG. 1, rotation of the pulley results in a rotation of the sensor housing relative to the main housing (e.g., in the example of FIG. 1, rotation of second pulley 128 causes the first pulley 120 to rotate due to a movement of belt 122, with first pulley 120 coupled to the sensor housing 108). In this way, each electrical pulse transmitted to the stepper motor corresponds to an amount of rotation of the sensor housing relative to the main housing. By adjusting the number of electrical pulses (e.g., increasing or decreasing the number of pulses) transmitted to the stepper motor during a single large-angle scan, a number of steps rotated by the stepper motor may be increased or decreased accordingly, thereby increasing or decreasing an amount of angle through which the sensor housing is rotated relative to the main housing during the large-angle scan. In other words, adjusting the operation of the large-angle adjustment element as described above increases or decreases an angle over the large-angle scan is performed, such that a greater amount of the region of interest may be included within the large-angle scan.

The method continues from 708 to 710 where the method includes determining whether small-angle volume compound imaging (VCI) is desired. For example, the operator of the ultrasound system may determine that small-angle VCI is desired in order to increase a clarity and/or contrast of images acquired through the region of interest during a large-angle scan. For example, during a large-angle scan (e.g., a scan spanning an angle of 45 degrees, for example, and performed via actuation of the large-angle adjustment element as described below), a first plurality of images may be acquired along a first plurality of imaging planes. Small-angle VCI may produce a second plurality of images along a second plurality of imaging planes during the large-angle scan via actuation of the small-angle adjustment element in combination with actuation of the large-angle adjustment element as described below with reference to 716. A portion of the second plurality of images may be compared and/or combined with a portion of the first plurality of images in order to produce a plurality of compound images associated with the first plurality of imaging planes and with increased clarity and/or contrast relative to the first plurality of images. Further examples are described below.

In another example, the operator of the system may determine that small-angle VCI is not desired. For example, the region of interest may be a region in which images produced by the ultrasound system typically do not include a high amount of acoustical artifacts, image noise, etc. As a result, the operator may determine that producing compound images via small-angle VCI is less desirable than acquiring non-compound images (e.g., images without VCI) at an increased rate.

If small-angle VCI is not desired at 710, the method continues to 712 where the method includes scanning the ROI by actuating the large-angle adjustment element and not actuating the small-angle adjustment element. In other words, a plurality of electrical pulses may be transmitted to the stepper motor in order to drive the stepper motor and rotate the sensor housing relative to the main housing through a large scan angle (e.g., 45 degrees, 50 degrees, etc.). As the sensor housing rotates relative to the main housing, the transducer array may transmit acoustic waves through the region of interest and receive echoes as described above with reference to FIG. 1 and FIG. 4. In this way, images associated with a plurality of imaging planes may be transmitted from the ultrasound probe to the workstation.

The method continues from 712 to 718 where the method includes displaying a compound and/or non-compound image based on scan results. In one example, each of the non-compound images acquired during the large-angle scan at 712 may be displayed by the display device in an order in which they were acquired. In another example, one or more of the non-compound images acquired during the large-angle scan may be displayed side-by-side. In yet another example, a plurality of the non-compound images may be compared and/or combined in order to form at least one compound image for display on the display device.

Returning to 710, if small-angle VCI is desired at 710, the method continues to 714 where the method includes adjusting a frequency and/or a shape of a signal to the small-angle adjustment element based on an amount of VCI desired. For example (and as described above with reference to 606 and 614 of method 600 shown by FIG. 6), adjusting an amplitude of an electrical signal transmitted to the piezoelectric actuator of the small-angle adjustment element may increase or decrease an amount of angle spanned during a small-angle scan (e.g., may increase or decrease a rotation of the transducer array relative to the sensor housing during the small-angle scan). In another example, adjusting a frequency and/or shape of the electrical signal may increase or decrease an amount of images acquired during a small-angle scan. For example, as described above with reference to FIG. 4, decreasing a frequency of the electrical signal may increase an amount of images acquired during a small-angle scan via actuation of the piezoelectric actuator.

The method continues from 714 to 716 where the method includes scanning the ROI by actuating both of the small-angle adjustment element and the large-angle adjustment element. For example, the large-angle adjustment element may be actuated to rotate the sensor housing relative to the main housing through a relatively large angle (e.g., 45 degrees, in one example) to perform a large-angle scan, thereby acquiring a first plurality of images associated with a first plurality of imaging planes. During the large-angle scan, the small-angle adjustment element may be actuated to rotate the transducer array relative to the sensor housing to acquire a second plurality of images. In one example, the second plurality of images may be associated with a second plurality of imaging planes different from the first plurality of imaging planes. For example, each imaging plane of the first plurality of imaging planes may be positioned between two adjacent imaging planes of the second plurality of imaging planes, such that the two adjacent imaging planes of the second plurality of imaging planes are at a relatively small angle (±2.5 degrees, for example) relative to the imaging plane of the first plurality of imaging planes. In this way, by actuating the small-angle adjustment element in combination with the large-angle adjustment element, an increased amount of images may be acquired for each step of the stepper motor.

The method continues from 716 to 718 where the method includes displaying a compound and/or a non-compound image based on scan results. In one example, each of the non-compound images acquired during the large-angle scan at 716 (e.g., the first plurality of images referenced by 716) may be compared and/or combined with the non-compound images acquired during at least one small-angle scan at 716 (e.g., the second plurality of images referenced by 716) in order to form a plurality of compound images. The plurality of compound images may be associated with the same plurality of imaging planes as the first plurality of images. In other words, the plurality of compound images may be similar to the first plurality of images (e.g., associated with the same imaging planes) and may have an increased clarity and/or contrast relative to the first plurality of images due to additional details provided by the second plurality of images. The compound images may then be displayed by the display device in an order in which they were acquired. In another example, one or more of the compound images may be displayed side-by-side by the display device.

A technical effect of the disclosure is to increase a scanning precision of an ultrasound probe by including a small-angle adjustment element configured to rotate a transducer array of the ultrasound probe relative to a sensor housing, as described above with reference to FIGS. 1-7. In one example, the small-angle adjustment element includes a piezoelectric actuator. The piezoelectric actuator may be selectively energized in order to adjust an amount of rotation of the transducer array relative to the sensor housing. An angle of an imaging plane produced by the transducer array may be adjusted in this way by a much smaller amount (e.g., 5 degrees) relative to an adjustment to the angle produced by rotating the sensor housing via actuation of a large-angle adjustment element (e.g., 45 degrees). In this way, an amount of angle between imaging planes may be reduced, and an amount of acoustical artifacts, image noise, etc. included within a resulting compound image formed by one or more of the non-compound images may be increased.

Another technical effect of the disclosure is to actuate the small-angle adjustment element and large-angle adjustment element together or separately in order to adjust the number and/or types of images displayed on a display device according to operator preference. For example, the operator may increase a number of images acquired during a small-angle scan in order to increase a clarity and/or contrast of one or more compound images produced from the images acquired during the small-angle scan. In another example, the operator may actuate the small-angle adjustment element during a large-angle scan in order to increase a clarity and/or contrast of images acquired during the large-angle scan. In this way, a quality of images produced by the ultrasound probe may be increased for a wide range of applications.

In one embodiment, an ultrasound probe includes: a sensor housing; a piezoelectric actuator disposed within the sensor housing; and a transducer array coupled with the sensor housing, the transducer array rotatable relative to the sensor housing via energization of the piezoelectric actuator. In a first example of the ultrasound probe, the ultrasound probe includes a shaft disposed within the sensor housing, the shaft coupled to the piezoelectric actuator and the transducer array. A second example of the ultrasound probe optionally includes the first example, and further includes wherein a first end of the piezoelectric actuator is coupled to the sensor housing and a second end of the piezoelectric actuator is coupled to the shaft. A third example of the ultrasound probe optionally includes one or both of the first and second examples, and further includes wherein a first end of the shaft is directly coupled to a bottom surface of the sensor housing and a second end of the shaft is coupled to the transducer array. A fourth example of the ultrasound probe optionally includes one or more or each of the first through third examples, and further includes a bearing rotatably coupled to a bearing housing and disposed within the bearing housing, the bearing housing coupled to a bottom surface of the sensor housing, and wherein a first end of the shaft is coupled to the bearing and a second end of the shaft is coupled to the transducer array. A fifth example of the ultrasound probe optionally includes one or more or each of the first through fourth examples, and further includes: a main housing, the sensor housing disposed in an interior of the main housing; a stepper motor and a plurality of pulleys disposed within the interior of the main housing, the plurality of pulleys coupled via a belt and rotatable via actuation of the stepper motor, with a first pulley of the plurality of pulleys coupled to the sensor housing and configured to rotate the sensor housing relative to the main housing.

In one embodiment, a method includes: transmitting a first electrical signal to a piezoelectric actuator of an ultrasound probe; and responsive to the first electrical signal, rotating a transducer array of the ultrasound probe by a first rotational angle by expanding or contracting the piezoelectric actuator. In a first example of the method, expanding the piezoelectric actuator rotates the transducer array in a first direction, and contracting the piezoelectric actuator rotates the transducer array in a second direction opposite to the first direction. A second example of the method optionally includes the first example, and further includes adjusting a frequency of the first electrical signal in order to adjust a rotational speed of the transducer array. A third example of the method optionally includes one or both of the first and second examples, and further includes wherein increasing the frequency increases the rotational speed, and decreasing the frequency decreases the rotational speed. A fourth example of the method optionally includes one or more or each of the first through third examples, and further includes adjusting an amplitude of the first electrical signal in order to adjust the first rotational angle of the transducer array. A fifth example of the method optionally includes or more or each of the first through fourth examples, and further includes wherein increasing the amplitude increases the first rotational angle, and decreasing the amplitude decreases the first rotational angle. A sixth example of the method optionally includes or more or each of the first through fifth examples, and further includes wherein adjusting the amplitude of the first electrical signal in order to adjust the first rotational angle of the transducer array includes increasing or decreasing the first rotational angle by a positive amount less than five degrees. A seventh example of the method optionally includes or more or each of the first through sixth examples, and further includes wherein expanding the piezoelectric actuator occurs responsive to a positive-valued voltage of the first electrical signal, and wherein contracting the piezoelectric actuator occurs responsive to a negative-valued voltage of the first electrical signal. An eighth example of the method optionally includes or more or each of the first through seventh examples, and further includes actuating a stepper motor of the ultrasound probe via a second electrical signal transmitted to the stepper motor, wherein actuating the stepper motor rotates a sensor housing including the transducer array by a second rotational angle, and wherein the second rotational angle is greater than the first rotational angle.

In another embodiment, an ultrasound probe includes: a main housing including a central axis; a stepper motor and a sensor housing disposed within an interior of the main housing, the sensor housing pivotable relative to the central axis via energization of the stepper motor; a transducer array pivotally coupled to the sensor housing via a shaft disposed within the sensor housing; and a piezoelectric actuator coupled to an interior wall of the sensor housing and the shaft, the transducer array being pivotable relative to the sensor housing via energization of the piezoelectric actuator. In a first example of the ultrasound probe, the ultrasound probe includes a controller electrically coupled with the ultrasound probe and including instructions stored in non-transitory memory to adjust a first electrical signal sent to the piezoelectric actuator and a second electrical signal sent to the stepper motor in response to input from an operator of the ultrasound probe. A second example of the ultrasound probe optionally includes the first example, and further includes wherein the sensor housing is not pivotable relative to the main housing via energization of the piezoelectric actuator, and wherein the transducer array is not pivotable relative to the sensor housing via energization of the stepper motor. A third example of the ultrasound probe optionally includes one or both of the first and second examples, and further includes wherein the stepper motor is configured to pivot the sensor housing by a first amount and the piezoelectric actuator is configured to pivot the transducer array by a second amount, with the second amount being less than the first amount relative to the central axis. A fourth example of the ultrasound probe optionally includes one or more or each of the first through third examples, and further includes wherein the sensor housing pivots along a rotational axis arranged perpendicular with the central axis.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An ultrasound probe comprising:
a sensor housing;
a piezoelectric actuator disposed within the sensor housing; and
a transducer array coupled with the sensor housing, the transducer array rotatable relative to the sensor housing via energization of the piezoelectric actuator.

2. The ultrasound probe of claim 1, further comprising a shaft disposed within the sensor housing, the shaft coupled to the piezoelectric actuator and the transducer array.

3. The ultrasound probe of claim 2, wherein a first end of the piezoelectric actuator is coupled to the sensor housing and a second end of the piezoelectric actuator is coupled to the shaft.

4. The ultrasound probe of claim 3, wherein a first end of the shaft is directly coupled to a bottom surface of the sensor housing and a second end of the shaft is coupled to the transducer array.

5. The ultrasound probe of claim 3, further comprising a bearing rotatably coupled to a bearing housing and disposed within the bearing housing, the bearing housing coupled to a bottom surface of the sensor housing, and wherein a first end of the shaft is coupled to the bearing and a second end of the shaft is coupled to the transducer array.

6. The ultrasound probe of claim 1, further comprising:
a main housing, the sensor housing disposed in an interior of the main housing;
a stepper motor and a plurality of pulleys disposed within the interior of the main housing, the plurality of pulleys coupled via a belt and rotatable via actuation of the stepper motor, with a first pulley of the plurality of pulleys coupled to the sensor housing and configured to rotate the sensor housing relative to the main housing.

7. A method comprising:
transmitting a first electrical signal to a piezoelectric actuator of an ultrasound probe; and
responsive to the first electrical signal, rotating a transducer array of the ultrasound probe by a first rotational angle by expanding or contracting the piezoelectric actuator.

8. The method of claim 7, wherein expanding the piezoelectric actuator rotates the transducer array in a first direction, and contracting the piezoelectric actuator rotates the transducer array in a second direction opposite to the first direction.

9. The method of claim 7, further comprising adjusting a frequency of the first electrical signal in order to adjust a rotational speed of the transducer array.

10. The method of claim 9, wherein increasing the frequency increases the rotational speed, and decreasing the frequency decreases the rotational speed.

11. The method of claim 7, further comprising adjusting an amplitude of the first electrical signal in order to adjust the first rotational angle of the transducer array.

12. The method of claim 11, wherein increasing the amplitude increases the first rotational angle, and decreasing the amplitude decreases the first rotational angle.

13. The method of claim 12, wherein adjusting the amplitude of the first electrical signal in order to adjust the first rotational angle of the transducer array includes increasing or decreasing the first rotational angle by a positive amount less than five degrees.

14. The method of claim 7, wherein expanding the piezoelectric actuator occurs responsive to a positive-valued voltage of the first electrical signal, and wherein contracting the piezoelectric actuator occurs responsive to a negative-valued voltage of the first electrical signal.

15. The method of claim 7, further comprising actuating a stepper motor of the ultrasound probe via a second electrical signal transmitted to the stepper motor, wherein actuating the stepper motor rotates a sensor housing including the transducer array by a second rotational angle, and wherein the second rotational angle is greater than the first rotational angle.

16. An ultrasound probe comprising:
a main housing including a central axis;
a stepper motor and a sensor housing disposed within an interior of the main housing, the sensor housing pivotable relative to the central axis via energization of the stepper motor;
a transducer array pivotally coupled to the sensor housing via a shaft disposed within the sensor housing; and
a piezoelectric actuator coupled to an interior wall of the sensor housing and the shaft, the transducer array being pivotable relative to the sensor housing via energization of the piezoelectric actuator.

17. The ultrasound probe of claim 16, further comprising a controller electrically coupled with the ultrasound probe and including instructions stored in non-transitory memory to adjust a first electrical signal sent to the piezoelectric actuator and a second electrical signal sent to the stepper motor in response to input from an operator of the ultrasound probe.

18. The ultrasound probe of claim 16, wherein the sensor housing is not pivotable relative to the main housing via energization of the piezoelectric actuator, and wherein the transducer array is not pivotable relative to the sensor housing via energization of the stepper motor.

19. The ultrasound probe of claim 16, wherein the stepper motor is configured to pivot the sensor housing by a first amount and the piezoelectric actuator is configured to pivot the transducer array by a second amount, with the second amount being less than the first amount relative to the central axis.

20. The ultrasound probe of claim 19, wherein the sensor housing pivots along a rotational axis arranged perpendicular with the central axis.
